SEQUENCE LISTING (3 sequences)

SEQ ID NO: 1 - protein, 605 aa, homo sapiens
MMDLELPPPGLPSQQDMDLIDILWRQDIDLGVSREVFDFSQRRKEYELEKQKKLEKERQEQLQKEQEKAFFAQLQLDEETGEFLPIQPAQHIQSETSGSANYSQVAHIPKSDALYFDDCMQLLAQTFPFVDDNEVSSATFQSLVPDIPGHIESPVFIATNQAQSPETSVAQVAPVDLDGMQQDIEQVWEELLSIPELQCLNIENDKLVETTMVPSPEAKLTEVDNYHFYSSIPSMEKEVGNCSPHFLNAFEDSFSSILSTEDPNQLTVNSLNSDATVNTDFGDEFYSAFIAEPSISNSMPSPATLSHSLSELLNGPIDVSDLSLCKAFNQNHPESTAEFNDSDSGISLNTSPSVASPEHSVESSSYGDTLLGLSDSEVEELDSAPGSVKQNGPKTPVHSSGDMVQPLSPSQGQSTHVHDAQCENTPEKELPVSPGHRKTPFTKDKHSSRLEAHLTRDELRAKALHIPFPVEKIINLPVVDFNEMMSKEQFNEAQLALIRDIRRRGKNKVAAQNCRKRKLENIVELEQDLDHLKDEKEKLLKEKGENDKSLHLLKKQLSTLYLEVFSMLRDEDGKPYSPSEYSLQQTRDGNVFLVPKSKKPDVKKN

SEQ ID NO: 2 - protein, 589 aa, homo sapiens
MDLIDILWRQDIDLGVSREVFDFSQRRKEYELEKQKKLEKERQEQLQKEQEKAFFTQLQLDEETGEFLPIQPAQHTQSETSGSANYSQVAHIPKSDALYFDDCMQLLAQTFPFVDDNEVSSATFQSLVPDIPGHIESPVFIATNQAQSPETSVAQVAPVDLDGMQQDIEQVWEELLSIPELQCLNIENDKLVETTMVPSPEAKLTEVDNYHFYSSIPSMEKEVGNCSPHFLNAFEDSFSSILSTEDPNQLTVNSLNSDATVNTDFGDEFYSAFIAEPSISNSMPSPATLSHSLSELLNGPIDVSDLSLCKAFNQNHPESTAEFNDSDSGISLNTSPSVASPEHSVESSSYGDTLLGLSDSEVEELDSAPGSVKQNGPKTPVHSSGDMVQPLSPSQGQSTHVHDAQCENTPEKELPVSPGHRKTPFTKDKHSSRLEAHLTRDELRAKALHIPFPVEKIINLPVVDFNEMMSKEQFNEAQLALIRDIRRRGKNKVAAQNCRKRKLENIVELEQDLDHLKDEKEKLLKEKGENDKSLHLLKKQLSTLYLEVFSMLRDEDGKPYSPSEYSLQQTRDGNVFLVPKSKKPDVKKN

SEQ ID NO: 3 - protein, 582 aa, homo sapiens
MDLIDILWRQDIDLGVSREVFDFSQRRKEYELEKQKKLEKERQEQLQKEQEKAFFAQLQLDEETGEFLPIQPAQHIQSETSGSANYSQVAHIPKSDALYFDDCMQLLAQTFPFVDDNESLVPDIPGHIESPVFIATNQAQSPETSVAQVAPVDLDGMQQDIEQVWEELLSIPELQCLNIENDKLVETTMVPSPEAKLTEVDNYHFYSSIPSMEKEVGNCSPHFLNAFEDSFSSILSTEDPNQLTVNSLNSDATVNTDFGDEFYSAFIAEPSISNSMPSPATLSHSLSELLNGPIDVSDLSLCKAFNQNHPESTAEFNDSDSGISLNTSPSVASPEHSVESSSYGDTLLGLSDSEVEELDSAPGSVKQNGPKTPVHSSGDMVQPLSPSQGQSTHVHDAQCENTPEKELPVSPGHRKTPFTKDKHSSRLEAHLTRDELRAKALHIPFPVEKIINLPVVDFNEMMSKEQFNEAQLALIRDIRRRGKNKVAAQNCRKRKLENIVELEQDLDHLKDEKEKLLKEKGENDKSLHLLKKQLSTLYLEVFSMLRDEDGKPYSPSEYSLQQTRDGNVFLVPKSKKPDVKKN

(19) United States
(12) Patent Application Publication
(10) Pub. No.: US 2016/0263108 A1
(43) Pub. Date: Sep. 15, 2016

(54) NRF2 INHIBITORS AND COMPOSITIONS FOR TREATING MYCOBACTERIAL INFECTIONS

(71) Applicant: THE HAMNER INSTITUTES, Research Triangle Park, NC (US)

(72) Inventors: M. Bud Nelson, Research Triangle Park, NC (US); Jinbo Pi, Research Triangle Park, NC (US); Melvin E. Anderson, Research Triangle Park, NC (US)

(21) Appl. No.: 14/912,465

(22) PCT Filed: Jul. 17, 2014
(86) PCT No.: PCT/US14/46936

§ 371 Date: Feb. 17, 2016

Related U.S. Application Data
(60) Provisional application No. 61/847,640, filed on Jul. 18, 2013.
(60) Provisional application No. 61/891,956, filed on Oct. 17, 2013.
(60) Provisional application No. 62/021,387, filed on Jul. 7, 2014.

(51) Int. Cl. A61K 31/496; A61K 31/44; A61K 31/16; A61K 31/166; A61K 45/06; A61K 31/4465; A61K 31/133; A61K 31/15; A61K 31/341; A61K 31/4965; A61K 31/4409

(52) U.S. Cl. CPC A61K 31/496; A61K 31/4965; A61K 31/44; A61K 31/16; A61K 31/166; A61K 31/4409; A61K 31/4465; A61K 31/133; A61K 31/15; A61K 31/341; A61K 45/06

(57) ABSTRACT

Provided herein are methods, compositions, and kits for Nr2 inhibitors and their use in treatment of mycobacterial infection and in combination therapy with known antitubercular drugs.

### FIELD OF THE INVENTION

The present invention relates to the discovery of compounds which can inhibit Nrf2 activity, and more particularly Nrf2-ARE activity, as well as relates to pharmaceutical compositions containing them as an active ingredient, and their use as medicaments in a method for treating mycobacterial infections and tuberculosis.

### BACKGROUND OF THE INVENTION

One of the most critical cytoprotective mechanisms against oxidative/electrophilic stress in vertebrates is the Keap1 (Kelch-like ECH protein 1)-Nrf2 (Nuclear factor E2-related factor 2) pathway. Nrf2, characterized by the amino acid sequence comprising SEQ ID NO:1 including isoforms thereof (see, e.g., SEQ ID NOs:2 & 3), recognizes a unique DNA sequence known as the antioxidant response element (ARE). Keap1 binds to Nrf2 in the cytoplasm of a cell, resulting in Nrf2 degradation. Thus, under normal homeostatic conditions, a low amount of cellular Nrf2 is mainly controlled by Keap1-mediated ubiquitination, and subsequent proteasomal degradation. However, following exposure to electrophiles or oxidative stress (including reactive oxygen species), Keap 1 is inactivated, and Nrf2 is stabilized (“Nrf2 activation”). Nrf2, as a potent transcription activator, translocates into the nucleus and activates (by binding ARE) transcription of a number of genes having functional ARE, including cytoprotective genes, such as encoding antioxidant enzymes, phase II detoxification enzymes, and multidrug resistant proteins. Thus, NRF2-mediated adaptive antioxidant response plays pivotal roles against oxidative/electrophilic stress, and in chemical detoxification. For example, Nrf2 has been shown to regulate the transcription of genes that encode drug metabolizing enzymes, including UGT (UDP-glucuronosyltransferase) and NQO1 (NAD(P)H quinone oxidoreductase 1), cytochrome P450 2A5 (CYP2A5), and glutathione S-transferase (GST). Nrf2 also regulates the expression of genes that are involved in direct reduction of reactive oxygen species (ROS), including superoxide dismutase, catalase, and glutathione peroxidases. Additionally, Nrf2 induces genes involved in reduction of oxidized proteins, such as thioredoxin-1, thioredoxin reductase-1, and sulfiredoxin, as well as genes encoding enzymes that synthesize glutathione (GSH); i.e., γ-glutamate-cysteine ligase catalyze subunit (Gclc) and the modifier subunit (Gclm). Lastly, Nrf2 (via ARE) is involved in the induction of genes encoding drug transporters, an important detoxification pathway which facilitates cellular excretion of conjugated drug metabolites.

With 9 million new cases and almost 2 million deaths per year, tuberculosis is an infectious disease of major medical impact. Current standard treatment for tuberculosis is with isoniazid, such as when used alone for six to nine months. Standard “short” course treatment for tuberculosis comprises isoniazid, rifampicin (also known as rifampin), pyrazinamide, and optionally ethambutol, for two months; then isoniazid and rifampicin alone for an additional four months. However, treatment has been complicated by the emergence of extensively drug resistant Mycobacteria tuberculosis which are resistant to the most effective first line therapeutic drugs (e.g., isoniazid and rifampin) as well as the most effective second line therapeutic drugs. There is a dearth of new drugs or new mechanisms of action which can be utilized in the development of treatments for tuberculosis; hence, effective treatments for extensively drug resistant M. tuberculosis have not been established. Thus, there is an urgent need for an innovative approach to produce new drugs for treating tuberculosis.

### SUMMARY OF THE INVENTION

Discovered are compounds, or compositions containing such compounds, that preferably downregulate or inhibit Nrf2 activity, including inhibiting Nrf2 transactivation of genes downstream from Nrf2, particularly genes having an ARE (antioxidant response element) in their promoter. These newly identified NRF2 inhibitors do not appear to reduce the level of mRNA and/or protein expression of NRF2, but rather suppress Nrf2 activity (e.g., affect Nrf2 function) which subsequently affects induction of ARE-driven gene expression (hence, Nrf2-ARE inhibitory activity).

An aspect of the present invention, based in part on the inventors' discovery that many antitubercular drugs surprisingly have Nrf2-ARE-inhibitory activity, is a method to treat tuberculosis or mycobacterial infection comprising administering a compound or composition to an individual in need thereof, wherein the compound (or composition comprising the compound): (a) inhibits Nrf2 activity (e.g., as can be assessed by Nrf2-ARE-inhibitory activity); (b) comprises a compound represented by Formula I or Formula II; and (c) is a compound other than a known antitubercular drug. Thus, the compound is represented by Formula I or Formula II with the proviso that the compound is not isoniazid, pyrazinamide, pyrazine-2-thio carboxamide, sparfloxacin, ethambutol dihydrochloride, ethionamide, amikacin, aminosalicylic acid, capreomycin, cycloserine, kanamycin, rifamycins (i.e., rifampin, rifapentine and rifabutin), streptomycin, thioacetazone, ofloxacin, ciprofloxacin, clarithromycin, azithromycin, bedaquiline, SQ 109, thioacetazone and fluoroquinolones or salt thereof), or other known antitubercular drug. The method for treatment may comprise preventing or treating an active, reactivation, or inactive M. tuberculosis infection. In one aspect the compound comprises a heterocyclic compound having a chemical side chain selected from the group consisting of a hydrazine and a carboxamide, and is represented by Formula I or Formula II with the proviso that the compound is not isoniazid, pyrazinamide (also known as pyrazine carboxamide), ethionamide, pyrazinamine, pyrazine-2-thio carboxamide, N-hydroxymethyl pyrazine thiocarboxamide, or N-substituted 3-aminopyrazine-2,5-dicarbonitriles.

In a preferred embodiment, an Nrf2 inhibitor is administrated with one or more additional therapeutic agents for treating M. tuberculosis infection in an individual in need thereof (e.g., the one or more additional therapeutic agents consisting of a known antitubercular drug including but not limited to isoniazid, pyrazinamide, pyrazinamine, pyrazine-2-thio carboxamide, N-hydroxymethyl pyrazine thiocarboxamide, N-substituted 3-aminopyrazine-2,5-dicarbonitriles, sparfloxacin, ethambutol dihydrochloride, ethionamide, amikacin, aminosalicylic acid, capreomycin, cycloserine, kanamycin, rifamycins, streptomycin, ofloxacin, ciprofloxacin, clarithromycin, azithromycin, bedaquiline, thioacetazone, SQ 109, and fluoroquinolones or salt thereof). Thus, employed in methods for treating (prophylactically and/or therapeutically) tuberculosis in an individual, the method comprising the step of administering to an individual infected with Mycobacterium tuberculosis a medically effective amount of an Nrf2 inhibitor by itself or in combination therapy with a known antitubercular drug, thereby treating tuberculosis infection. Also provided is a pharmaceutical composition or medicament comprising (a) one or more Nrf2 inhibitors comprising a compound represented by Formula I or Formula II with the proviso that the compound is not an antitubercular drug known for treating tuberculosis (known antitubercular drugs include but are not limited to, isoniazid, pyrazinamide, pyrazinamine, pyrazine-2-thio carboxamide, N-hydroxymethyl pyrazine thiocarboxamide, N-substituted 3-aminopyrazine-2,5-dicarbonitriles, sparfloxacin, ethambutol dihydrochloride, ethionamide, amikacin, aminosalicylic acid, capreomycin, cycloserine, kanamycin, rifamycins, streptomycin, thioacetazone, ofloxacin, ciprofloxacin, clarithromycin, azithromycin, bedaquiline, SQ 109, thioacetazone and fluoroquinolones or salt thereof), and (b) one or more known antitubercular drugs. The pharmaceutical composition or medicament may further comprise a pharmaceutically acceptable carrier.

Other aspects, objects and features of the invention will be apparent from the following description.

### BRIEF DESCRIPTION OF THE DRAWINGS

FIG. 1A is a graph showing that isoniazid (“INH”) suppresses ARE activity in 3T3-L1 preadipocytes in a concentration-dependent manner under basal (“Veh”) conditions, and that treatment with an Nrf2 activator (iAs³⁺) modulated the inhibition of ARE activity by isoniazid.

FIG. 1B is a graph showing that isoniazid (“INH”, 10 mM) suppresses Nrf2-ARE-dependent gene expression of glutamate-cysteine ligase catalytic subunit (“Gclc”) in 3T3-L1 preadipocytes under basal (“Veh”) conditions, and that treatment with an Nrf2 activator (tBHQ, 50 μM) modulated the inhibition of Nrf2-ARE activity by isoniazid. FIG. 1C is a graph showing that isoniazid (“INH”, 10 mM) suppresses ARE-dependent gene expression of NAD(P)H dehydrogenase [quinone] 1 (“Ngo1”) activity in 3T3-L1 preadipocytes under basal (“Veh”) conditions, and that treatment with an Nrf2 activator (tBHQ, 50 μM) modulated the inhibition of Nrf2-ARE activity by isoniazid.

FIG. 1D is a graph showing that isoniazid (“INH”, 10 mM) suppresses Nrf2-ARE-dependent gene expression of Heme oxygenase (“Ho1”) activity in 3T3-L1 preadipocytes under basal (“Veh”) conditions, and that treatment with an Nrf2 activator (tBHQ, 50 μM) modulated the inhibition of Nrf2-ARE activity by isoniazid.

FIG. 2A is a graph showing that isoniazid (“INH”) inhibits Nrf2-ARE activity in human hepatocellular liver carcinoma HepG2 cells in a concentration-dependent manner under basal (“Veh”) conditions, and that treatment with an Nrf2 activator (iAs³⁺) modulated the inhibition of Nrf2-ARE activity by isoniazid.

FIG. 2B is a graph showing that isoniazid (“INH”) suppresses Nrf2-ARE-dependent gene expression of Heme oxygenase (“Ho1”) activity in HepG2 cells in a concentration-dependent manner under basal (“Veh”) conditions, and that treatment with an Nrf2 activator (iAs³⁺) modulated the inhibition of Nrf2-ARE activity by isoniazid.

FIG. 3 is a graph showing that ethionamide (ETH) suppresses Nrf2-ARE activity in HepG2 cells in a concentration-dependent manner under basal (“Vehicle”) conditions, and that treatment with an Nrf2 activator (iAs³⁺) modulated the inhibition of Nrf2-ARE activity by ethionamide.

FIG. 4A is a graph showing that ethionamide (ETH) suppresses Nrf2-ARE-dependent gene expression (mRNA expression as a percent of the Control with Vehicle) of Heme oxygenase (“HO1”) activity in THP-1 cells in a concentration-dependent manner under basal (“Veh”) conditions, and that treatment with an Nrf2 activator (iAs³⁺) modulated the inhibition of Nrf2-ARE activity by ethionamide.

FIG. 4B is a graph showing that ethionamide (ETH) suppresses Nrf2-ARE-dependent gene expression (mRNA expression as a percent of the Control with Vehicle) of glutamate-cysteine ligase catalytic subunit (“GCLM”) activity in THP-1 cells in a concentration-dependent manner under basal (“Veh”) conditions, and that treatment with an Nrf2 activator (iAs³⁺) modulated the inhibition of Nrf2-ARE activity by ethionamide.

FIG. 4C is a graph showing that ethionamide (ETH) suppresses Nrf2-ARE-dependent gene expression (mRNA expression as a percent of the Control with Vehicle) of sulfiredoxin (“SRX”) activity in THP-1 cells in a concentration-dependent manner under basal (“Veh”) conditions, and that treatment with an Nrf2 activator (iAs³⁺) modulated the inhibition of Nrf2-ARE activity by ethionamide.

FIG. 5 is an illustration of chemical structures of compounds identified as inhibitors of Nrf2-ARE activity.

### DETAILED DESCRIPTION OF THE INVENTION

The present invention shows that compounds (including drugs and compositions comprising such compound), having a similar chemical structure or “chemical signature” as some known antitubercular drugs, have Nrf2-ARE-inhibitory activity; and may also be used in the treatment of M. tuberculosis infection. In one aspect, the compound of the invention is an Nrf2 inhibitor comprising a heterocyclic compound having a chemical side chain comprising a hydrazine, a carboxamide, or a combination thereof, and is represented by Formula I or Formula II, with the proviso that the compound is not isoniazid, pyrazinamide (also known as pyrazine carboxamide), ethionamide, pyrazinamine, pyrazine-2-thio carboxamide, N-hydroxymethyl pyrazine thiocarboxamide, N-substituted 3-aminopyrazine-2,5-dicarbonitriles, or other known antitubercular drug.

In regards to a role of Nrf2 in tuberculosis, it has been reported that Nrf2-deficient mice infected with M. tuberculosis have a significant reduction in granuloma formation and tubercule bacilli counts as compared with M. tuberculosis-infected mice that are not Nrf2-deficient, suggesting that Nrf2 activation is important in M. tuberculosis-induced granuloma formation, and that decreased Nrf2 activity could contribute to inhibition of M. tuberculosis infection. Thus, the present invention relates to the use of an Nrf2 inhibitor alone, or in combination with a known antitubercular drug, to treat M. tuberculosis infection.

While the terms used in the description of the invention are believed to be well understood by one of ordinary skill in the pharmaceutical arts, definitions, where provided herein, are set forth to facilitate description of the invention, and to provide illustrative examples for use of the terms.

As used herein, the terms “a”, “an”, and “the” mean “one or more”, unless the singular is expressly specified (e.g., singular is expressly specified, for example, in the phrase “a single formulation”).

The term “Nrf2 inhibitor” is used herein, for purposes of the specification and claims, to mean a chemical compound that (a) downregulates or inhibits Nrf2 activity, including inhibiting Nrf2 transactivation of genes downstream from Nrf2 (hence, has Nrf2-ARE-inhibitory activity); (b) is represented by Formula I or Formula; and (c) is a compound or drug other than (i.e., excludes) a known antitubercular drug (e.g., isoniazid, pyrazinamide, pyrazinamine, pyrazine-2-thio carboxamide, N-hydroxymethyl pyrazine thiocarboxamide, N-substituted 3-aminopyrazine-2,5-dicarbonitriles, sparfloxacin, and ethambutol dihydrochloride, ethionamide, amikacin, aminosalicylic acid, capreomycin, cycloserine, kanamycin, rifamycins (i.e., rifampin, rifapentine and rifabutin), streptomycin, ofloxacin, ciprofloxacin, clarithromycin, azithromycin, bedaquiline, and fluoroquinolones, SQ 109, thioacetazone, or salt thereof) or other known antitubercular drug. As shown herein, in one example, an Nrf2 inhibitor comprises a heterocyclic compound having a hydrazide moiety or carboxamide moiety (typically, as a side chain), and is selected from a compound represented by Formulas I-II.

In one aspect of the invention, an Nrf2 inhibitor is selected from compounds represented by Formula I, and a pharmaceutically acceptable salt thereof.

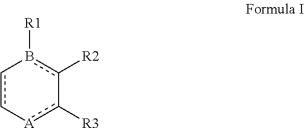

wherein:

### A is N or C;

### B is N or C;

R1 or R2 or R3 are each independently selected from H, (C₁-C₆)alkyl, CONH₂, CONHNH₂, CSNH₂, SO₂NH₂, NH₂, NHNH₂, CHCHCONH₂, CHCHCONHNH₂, or COCH₃;
wherein at least one of R1, R2, and R3 is selected from CONH₂, CONHNH₂, CSNH₂, SO₂NH₂, NH₂, NHNH₂, CHCHCONH₂, or CHCHCONHNH₂; and
the dashed lines represent optional double bonds; with the proviso that the compound of Formula I is not a known antitubercular drug.

In one aspect of the invention, an Nrf2 inhibitor is selected from compounds represented by Formula II, and a pharmaceutically acceptable salt thereof.

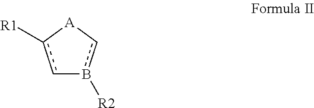

wherein:

### A is O or N;

### B is N or C;

R1 is selected from CONH₂, CONHNH₂, CSNH₂, SO₂NH₂, NH₂, NHNH₂, CHCHCONH₂, or CHCHCONHNH₂;
R2 is absent if B is NH;
if B is C, R2 is absent or selected from CH₃, CH₂CH₃, NH₂, or NHNH₂; and
the dashed lines represent optional double bonds; with the proviso that the compound of Formula II is not a known antitubercular drug.

From chemical libraries or collections of chemical compounds, a chemical compound can be screened for identifying an Nrf2 inhibitor by using assays for determining inhibition of Nrf2-ARE activity as known in the art, including those described herein in Example 1, as well as selecting compounds to test in the assays which have a chemical signature of Nrf2-ARE-inhibitory activity according to the invention (e.g. a compound represented by Formula I or Formula II). A preferred Nrf2 inhibitor may be used to the exclusion of an Nrf2 inhibitor other than the preferred Nrf2 inhibitor. In a method of combination therapy according to the invention, one or more Nrf2 inhibitors may be used in combination with one or more known antitubercular drugs in treating M. tuberculosis infection in an individual selected for treatment. Effectiveness in treating M. tuberculosis or other mycobacterial infection can be determined routinely using any one or more of standard in vivo models for mycobacterial infection known in the art. The Nrf2 inhibitor may be non-naturally occurring, or naturally occurring; and may further be isolated or purified. The Nrf2 inhibitor may be selected from a naturally occurring compound or a non-naturally occurring compound. For example, in one aspect, when an Nrf2 inhibitor is administered by itself (e.g., is the sole therapeutic agent in a pharmaceutical composition, and is not combined with at least one known antitubercular drug or second Nrf2 inhibitor, to produce a pharmaceutical composition), such Nrf2 inhibitor may be selected from a non-naturally occurring compound. In another example, in one aspect, when an Nrf2 inhibitor is administered in combination with one or more of a second Nrf2 inhibitor or known antitubercular drug (e.g., administered as a pharmaceutical composition comprising a combination of the Nrf2 inhibitor and one or more of a second Nrf2 inhibitor or known antitubercular drug), such Nrf2 inhibitor may be selected from a non-naturally occurring compound or a naturally occurring compound.

The term “non-naturally occurring” used in reference to a compound means that the compound is not known to exist in nature or that does not exist in nature. The term “naturally occurring” when used in connection with compounds refers to a compound which is found in nature. It is apparent to those skilled in the art that a naturally occurring compound can be modified or engineered by a human or by an engineered organism to be structurally or chemical different to form a non-naturally occurring compound.

The terms “purified” or “isolated” for a compound or composition refers to the physical state of the compound or composition following isolation from a synthetic process or purification step described herein or well known to those in the art, and in sufficient purity to be characterizable by standard analytical methods described herein or well known in the art.

The term “known antitubercular drug” is used herein to refer to compounds that have been shown to exhibit antibacterial activity against mycobacteria as known to those skilled in the art, or approved for therapeutic use as drugs for treating mycobacterial infections or tuberculosis caused by mycobacterial infections in humans or animals. Known antitubercular drugs also include a drug that exhibits antibacterial activity against M. tuberculosis or has a primary mechanism of antibacterial action other than Nrf2-ARE-inhibition, as understood by or known to those skilled in the art without utilization of the present invention. Known antitubercular drugs include but are not Imited to isoniazid, pyrazinamide, pyrazinamine, pyrazine-2-thio carboxamide, N-hydroxymethyl pyrazine thiocarboxamide, N-substituted 3-aminopyrazine-2,5-dicarbonitriles, sparfloxacin, ethambutol dihydrochloride, ethionamide, amikacin, aminosalicylic acid, capreomycin, cycloserine, kanamycin, rifamycins (i.e., rifampin, rifapentine and rifabutin), streptomycin, ofloxacin, ciprofloxacin, clarithromycin, azithromycin, bedaquiline, SQ 109, thioacetazone, fluoroquinolones, or a salt thereof. With respect to primary known mechanism of therapeutic action, the antitubercular drugs that exhibit Nrf2-ARE-inhibitory activity are drugs that have been shown to mediate a therapeutic effect by a mechanism other than Nrf2-ARE-inhibition. For example, ethionamide is an antitubercular agent that inhibits mycolic acid synthesis; isoniazid inhibits the synthesis of mycolic acids, an essential component of the bacterial cell wall; pyrazinamide inhibits membrane transport function at acid pH in Mycobacterium tuberculosis, as well as inhibits the activity of purified FAS fatty acid synthase; rifampin is a broad spectrum antibacterial that suppresses the initiation of RNA synthesis by binding to DNA-dependent RNA polymerase and inhibiting its activity; and sparfloxacin inhibits DNA gyrase which is needed for DNA topology, replication, repair, deactivation, and transcription. Thus, the primary or known mechanisms of therapeutic action of antitubercular drugs is generally known or considered to be by a mechanism other than Nrf2-ARE-inhibitory activity.

The term “individual” is used herein to mean a mammal, and more preferably, a human. The term “individual having an M. tuberculosis infection” or “individual infected with M. tuberculosis” are used interchangeably herein to mean a mammal, and more preferably a human, infected with M. tuberculosis. The infection may be inactive (latent, M. tuberculosis infection without manifested disease symptoms), reactivated, or active (M. tuberculosis infection with manifested disease symptoms). The infection may also comprise a multi-drug resistant strain of M. tuberculosis. Diagnosis of M. tuberculosis infection, or tuberculosis, is commonly achieved using a skin test, which involves intradermal exposure to tuberculin PPD (protein-purified derivative); wherein a measurable induration at the injection site by 48-72 hours after injection indicates exposure to mycobacterial antigens. Confirmation of M. tuberculosis infection can also be achieved using one or more additional methods known in the art including, but not limited to, body fluid (sputum, gastric washings, laryngeal swab, bronchoalveolar lavage, bronchial washings) smears and cultures for acid-fast bacilli, and polymerase chain reaction or gene probe tests for detecting M. tuberculosis.

The terms “first”, “second”, and “additional”, are used herein for purposes of distinguishing between two compounds, or between two or more compositions or drugs, as will be clearer from the description.

The phrase “medically effective amount” generally means an amount of a composition or compound that treats the particular disease, condition or disorder; ameliorates, relieves, or decreases one or more symptoms associated with the particular disease, condition or disorder, or treatment; or delays or prevents the onset of symptoms of, or a process associated, with the particular disease, condition or disorder, or treatment. More specifically, a “medically effective amount” of an Nrf2 inhibitor means an amount of the Nrf2 inhibitor effective to inhibit Nrf2-ARE activity, as can be determined by methods known in the art (as will also be apparent from the description and figures herein).

The term “pharmaceutically acceptable carrier” is used herein to mean any compound or composition or carrier medium useful in any one or more of administration, delivery, storage, stability of a composition or compound described herein. These carriers are known in the art to include, but are not limited to, a diluent, water, saline, suitable vehicle (e.g., liposome, microparticle, nanoparticle, emulsion, capsule), buffer, medical parenteral vehicle, excipient, aqueous solution, suspension, solvent, emulsions, detergent, chelating agent, solubilizing agent, salt, colorant, polymer, hydrogel, surfactant, emulsifier, adjuvant, filler, preservative, stabilizer, oil, binder, disintegrant, absorbant, flavor agent, and the like as broadly known in the pharmaceutical art.

The terms “treat”, “treats”, or “treating”, as used herein, embrace one or more of preventative (prophylactically) or therapeutically (palliative).

The terms “salt” or pharmaceutically acceptable salt”, as used herein, refers to inorganic or organic salts of a compound. These salts can be prepared, for example, by reacting a compound comprising an Nrf2 inhibitor, such as a compound represented by Formula I or Formula II, with an amount of acid or base, such as an equivalent amount, and in a medium such as one in which the salt formed then precipitates, or in an aqueous medium followed by lyophilization. Representative salts include bisulfate, sulfate, benzene sulfonate, camphorsulfonate, laurylsulphonate, methanesulfonate, toluenesulfonate, naphthalenesulformate, acetate, trifluoracetate, benzoate, borate, butyrate, citrate, formate, fumarate, hydrobromide, hydrochloride, hydroiodide, lactate, laurate, maleate, malonate, mesylate, nitrate, oxalate, phosphate, hexafluorophosphate, propionate, salicylate, stearate, succinate, tartrate, thiocyanate, and the like. The salts may include base salts based on the alkali and alkaline earth metals, such as calcium, sodium, lithium, magnesium, and potassium; or with organic bases such as with organic amines (e.g., dicyclohexylamine, t-butyl amine, methylamine, dimethylamine, triethylamine, ethylamine, procaine, morpholine, N-methylpiperidine, dibenzylamine, and the like); or as an ammonium salt.

A medically effective amount of a compound used in the invention, or a composition comprising a compound used in the invention, will depend on such factors as the mode of administration, the formulation for administration, disease to be modulated, the size and health of the individual to receive such a composition, and other factors which can be taken into consideration by a medical practitioner whom is skilled in the art of determining appropriate dosages for treatment. An amount of compound used in the invention in a composition to be administered may vary from 0.01 milligrams to about 500 milligrams, and more typically from about 1 milligram per day to about 300 milligram per day. In another example, the amount of a compound according to the invention to be administered is an amount which results in a blood concentration of from about 0.01 mM to 50 mM in an individual receiving the compound. One skilled in the art can apply known principles and models of drug delivery and pharmacokinetics to ascertain a likely range of dosages to be tested in preclinical and clinical studies for determining a medically effective amount of a compound used in the invention. A pharmaceutically acceptable carrier, used in a composition according to the invention, may facilitate one or more of storage, stability, administration, and delivery, of the composition. The carrier may be particulate, so that the composition may be in, for example, powder or solid form. The carrier may be in a semi-solid, gel, or liquid formula, so that the composition may be ingested, injected, applied, or otherwise administered. The carrier may be gaseous, so that the composition may be inhaled.

For oral administration of a composition containing a compound according to the invention, suitable formulations may be presented in the form of tablets, caplets, capsules, and the like, in which typically the compound of the invention may be present in a predetermined amount as a powder, granules, solution, or suspension as the sole active agent, or in combination with an additional one or more pharmaceutical agents. As known in the art, such oral formulations typically involve one or more of a binder (e.g., syrup, sorbitol, gum, corn starch, gelatin, acacia), a filler (e.g., lactose, sugar, starch, calcium phosphate), an excipient (e.g., dicalcium phosphate), a disintegrating agent (e.g., vegetable starch, alginic acid), a lubricant (e.g., magnesium stearate), a flavoring agent (sweetening agent, natural or artificial flavors). Such oral formulations may be coated or uncoated to modify their disintegration and/or absorption. Coating may be performed using conventional coating agents and methods known in the art.

The mode of administration of a compound or composition according to the invention to an individual (such as a human) in need of such composition or compound may be any mode known in the art to be suitable for delivering a pharmaceutical composition, and particularly suitable for counteracting or modulating the Nrf2-ARE-inhibitory effect of an Nrf2 inhibitor in cells, and may include but is not limited to, intravenously, intraperitoneally, orally, subcutaneously, intramuscularly, intranasally, transdermally, by perfusion, and by peristaltic techniques. Provided herein is combination therapy comprising administering to an individual one or more Nrf2 inhibitors with one or more known antitubercular drugs. In such combination therapy, an Nrf2 inhibitor and a known antitubercular drug may be administered concurrently, sequentially, or in regimen alternating between an Nrf2 inhibitor and a known antitubercular drug. Such combination therapy may optionally include one or more pharmaceutically acceptable carriers. The structure of an Nrf2 inhibitor as provided herein and a known antitubercular drug, and their generic or trademark names, are readily available to those skilled in the art, such as from the standard compendium of drugs (e.g., The Merck Index) or from the applicable pharmaceutical company's web site, as well as dosages applicable for treatment (see also The Physician's Desk Reference). Alternatively, the doses and dosage regimen of an Nrf2 inhibitor and a known antitubercular drug used in accordance with the invention in combination therapy, can be determined by a physician, taking into account the medical literature, the health, age and sex of the patient, the disease or condition or disorder to be treated, the mode of administration and dosing schedule, and other relevant considerations. Generally, dosages of such compounds can range from about 0.1 mg to 1000 mg per day, with more specific dosages dependent on the aforementioned factors.

Accordingly, also provided herein is a pharmaceutical composition or medicament comprising a medically effective amount of one or more Nrf2 inhibitors, in combination with a medically effective amount of one or more known antitubercular drugs; and optionally further comprising a pharmaceutically acceptable carrier.

### Example 1

In this Example, illustrated is the identification of a novel class of compounds with strong inhibitory effects on NRF2 activity including downstream genes (ARE activity). These compounds include a panel of antitubercular agents, such as isoniazid, ethionamide, ethambutol dihydrochloride, rifampicin, ethionamide, and sparfloxacin; and other compounds represented by Formula I or Formula II, particularly heterocyclic compounds having a hydrazide side chain or carboxamide side chain, including 4-aminobenzoic hydrazide, aminopyrazine, cyclohexanecarboxamide, 2-furoic hydrazide, phenylhydrazine, phenylacetic hydrazide, pyrazinecarboxamide, p-toluic hydrazide, 4-(aminomethyl)piperidine; isonicotinamide, and 2-amino-isonicotinamide (see, e.g., Table 1). These compounds decrease ARE-luciferase activity, in a concentration-dependent manner in treated cells, under basal and arsenite-treated conditions. These inhibitors suppress Nrf2-ARE activity, and thereby suppress induction of ARE-driven gene expression.

These chemical modulators of Nrf2-ARE activity were identified by a series of chemical screenings using an assay in which an ARE-luciferase reporter is stably expressed in cells in which there is confirmed constitutive activation of Nrf2-ARE activity. These cells include mouse preadipocyte 3T3-L1 cell line; mouse insulinoma MIN6 cell line; human keratinocyte HaCaT cell line; and human hepatocellular cancer cell line, HepG2 cells. A commercially available ARE-luciferase reporter, in ready-to-transduce lentiviral particles, was used for assessing when the Nrf2 pathway is activated or inhibited by a drug or chemical, via detection of any modulation of luciferase reporter activity which can then be measured quantitatively. This type of assay has been validated in the art to measure and represent induction or inhibition of Nrf2-ARE activity taking place in cells in the body.

Lentiviral transduction of 3T3-L1, HaCaT and HepG2 cells was performed based on manufacturer's protocol. Briefly, 24 hours before transduction, the cells to be transduced were plated in 6-well plates at 40-50% confluency in complete cell culture medium. The following day, hexadimethrine bromide, a transduction enhancer, was added to each well at a concentration of 8 μg/ml, and viral particles were added to each well at a concentration of 2×10⁵ transducing units/ml. After overnight incubation, medium containing viral particles was removed and replaced with fresh medium containing 2 μg/ml puromycin. Cells were grown to ˜90% confluence and sub-cultured in medium containing puromycin. The 3T3-L1 cells, HaCaT cells, MIN6 cells, and HepG2 cells, with stable expression of ARE-luciferase reporter, were used to identify ARE activators and inhibitors, and more particularly, inhibitors of Nrf2-ARE activity.

Assessed was the ability of a chemical compound (including drugs) to inhibit Nrf2-ARE activity under basal conditions (i.e., no added exogenous Nrf2 activator; absence of exogenous Nrf2 activator). For those chemicals identified as having Nrf2-ARE-inhibitory activity, also assessed was the ability of an Nrf2 activator to modulate the Nrf2-ARE-inhibitory activity of the chemical (e.g., induce Nrf2 activation which then lessens, prevents or inhibits (in “modulating”) Nrf2 inhibition). Briefly, chemicals were individually added to the cells, and incubated for 24 hours under basal conditions (no added Nrf2 activator), or with tBHQ-treated or sodium arsenite-treated cells (6 hour treatment, 5-10 μM iAs³⁺ or 50 μM tBHQ, each a known Nrf2 activator), and measured was luciferase activity, as compared to assay controls. The luciferase activity was measured by a commercially available luciferase reporter assay system according to the manufacturer's protocol. The luciferase activity was normalized to protein content or cell viability. To confirm an inhibitory effect on ARE as observed by a decrease in luciferase activity (as compared to the assay control), the chemical was also tested for its ability to inhibit, in a concentration dependent manner, cell expression of multiple ARE-dependent genes, including one or more of HO1 (Heme oxygenase 1), GCLC (Glutamate-cysteine ligase catalytic subunit), GCLM (Glutamate-cysteine ligase modifier subunit), Nqo1 (NAD(P)H dehydrogenase [quinone] 1) and SRX (sulfiredoxin 1) by using real-time quantitative reverse transcription polymerase chain reaction (RT-qPCR) and commercially available primers. An inhibitory effect on expression of multiple ARE-dependent genes was used as an indication of inhibition of Nrf2 activity. An increase in ARE-luciferase activity in cells treated with combination therapy comprising an Nrf2 activator and a drug having Nrf2-ARE-inhibitory activity, as compared to the ARE-luciferase activity in cells treated only with drug having Nrf2-ARE-inhibitory activity (e.g., in absence of exogenous Nrf2 activator), is indicative of the ability of an Nrf2 activator to modulate the Nrf2-ARE-inhibitory activity of a drug or compound having Nrf2-ARE-inhibitory activity.

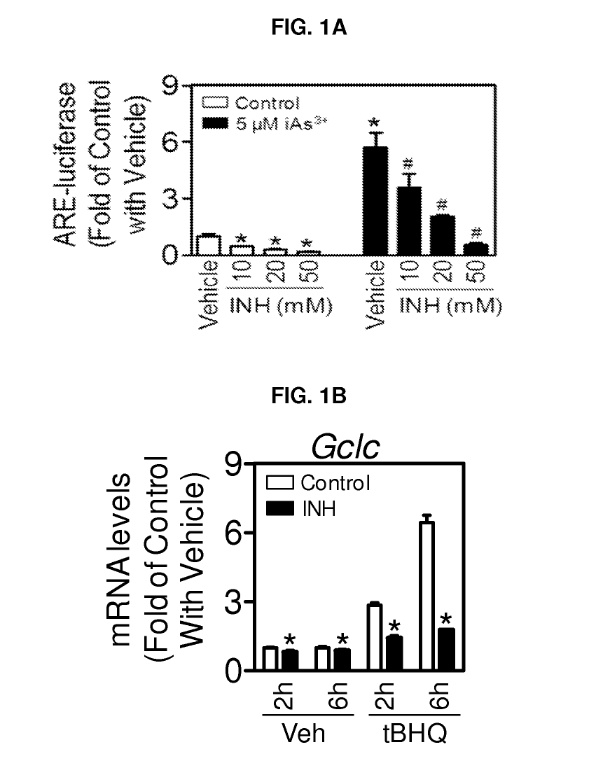

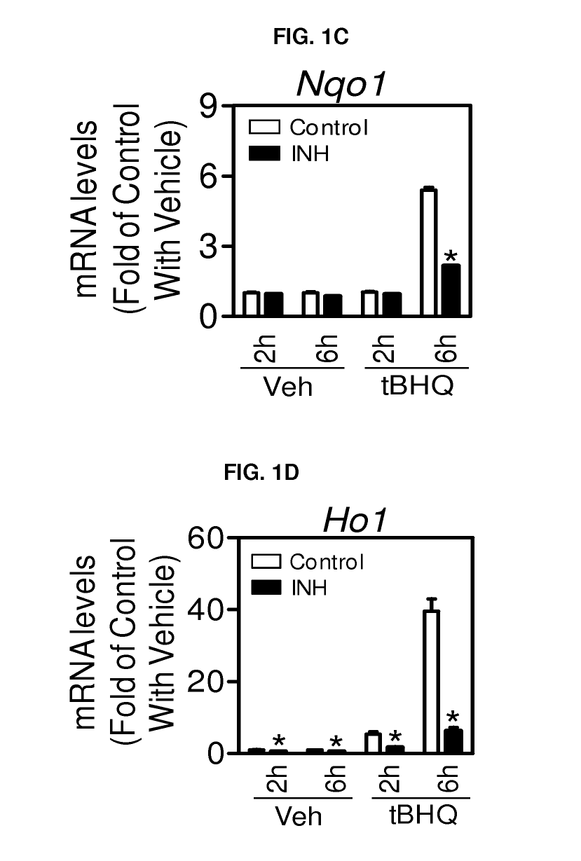

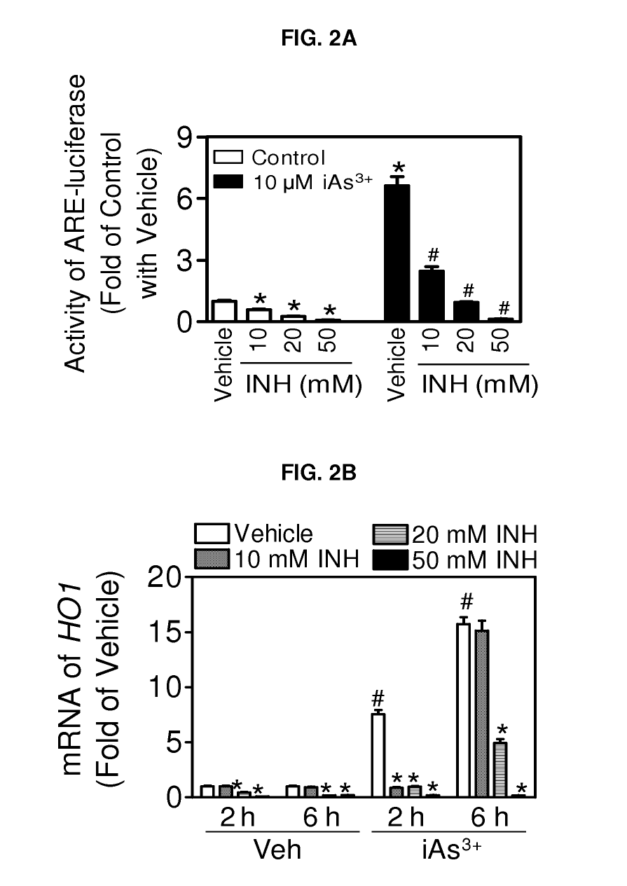

First, cytotoxicity of isoniazid (a widely used antitubercular drug) in 3T3-L1 cells and HepG2 cells was determined by exposing the cells to various concentrations of isoniazid, ranging from 1 mM to 200 mM, for 24 hours, and subsequently determining cell viability by a commercially available MTT (3-(4,5-dimethylthiazol-2-yl)-2,5-diphenyltetrazolium bromide) assay. From this determination, non-cytotoxic concentrations of isoniazid ranging from 1 mM to 50 mM were tested, as was an equal volume of cell culture without isoniazid (“Vehicle”) for comparison, in the ARE-luciferase reporter assay. As shown in FIGS. 1A and 2A, isoniazid (“INH”) exhibited a concentration-dependent inhibitory effect on ARE-luciferase activity in 3T3-L1 cells and HepG2 cells, respectively, under basal conditions. However, as shown in FIGS. 1A and 2A, cells treated with both isoniazid and Nrf2 activator (iAs³⁺-treated) exhibited substantial ARE-luciferase activity, indication that the Nrf2 activator was capable of modulating the Nrf2-ARE-inhibitory effect of isoniazid. The Nrf2-ARE-inhibitory effect of isoniazid was also observed in HaCaT cells stably expressing the same ARE-luciferase reporter assay. The inhibitory activity of isoniazid was confirmed by decreased expression of multiple ARE-dependent genes, including GCLC (FIG. 1B), NQO1 (FIG. 1C) and HO1 (FIG. 1 D, and FIG. 2B) under basal conditions. The ability of an Nrf2 activator (as illustrated by tert-butylhydroquinone (tBHQ) or iAs³⁺) to modulate the Nrf2-ARE-inhibitory activity of isoniazid was confirmed by an increased expression of multiple ARE-dependent genes, including GCLC (FIG. 1B), NQO1 (FIG. 1C) and HO1 (FIG. 1 D, and FIG. 2B) in cells treated with both the Nrf2 activator and the drug having Nrf2-ARE-inhibitory activity, as compared to expression of the ARE-dependent genes in cells treated with the drug having Nrf2-ARE-inhibitor activity alone (e.g., under basal conditions).

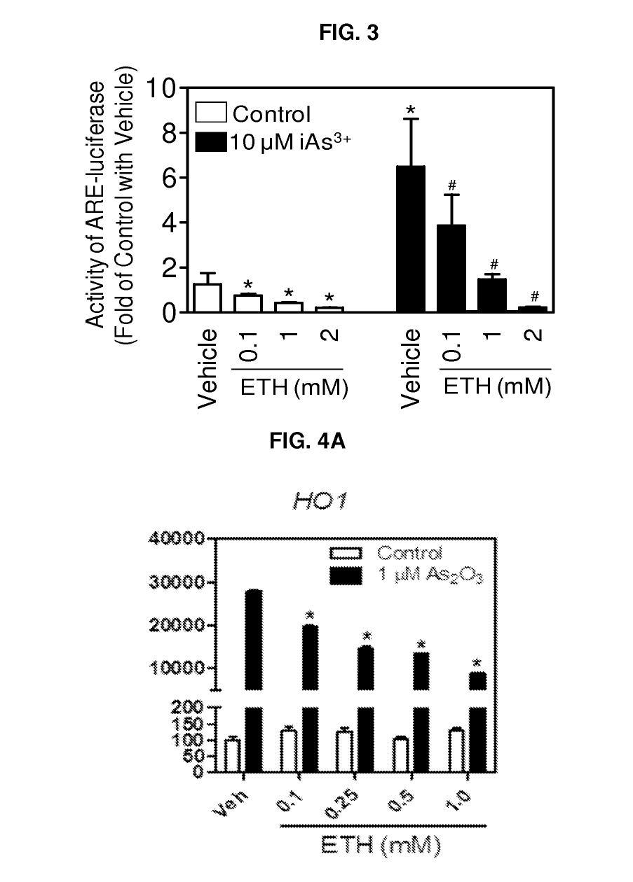

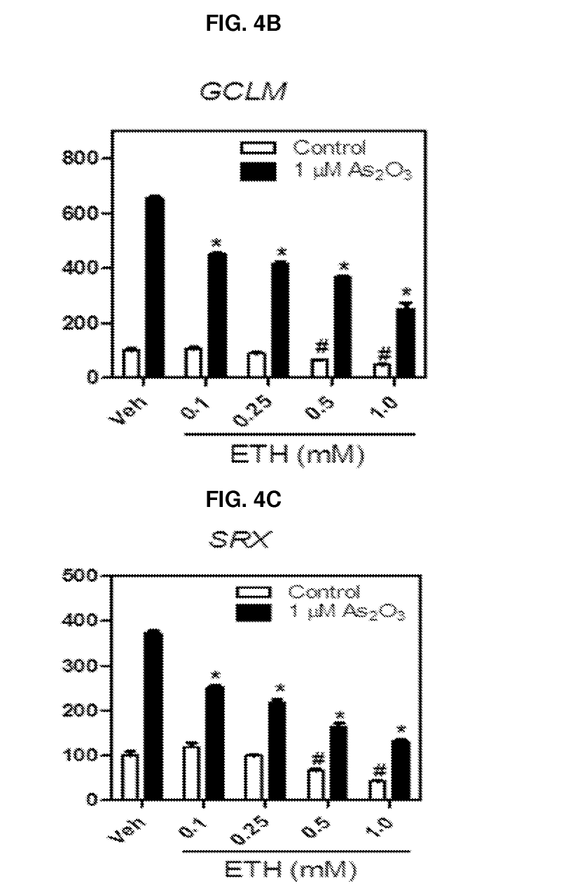

Using these methods and the ARE-luciferase reporter assay described herein, another antitubercular agent ethionamide (ETH), in non-cytotoxic concentrations, also displayed a concentration-dependent inhibitory effect on ARE-luciferase activity under basal conditions in HepG2 cells (FIG. 3). Additionally, as shown in FIG. 3, HepG2 cells treated with both ethionamide (ETH) and an Nrf2 activator (“iAs³⁺”) exhibited substantial ARE-luciferase activity, indicating that the Nrf2 activator was capable of modulating the Nrf2-ARE-inhibitory effect of the drug having Nrf2-ARE-inhibitory activity. Confirmation of the Nrf2-ARE-inhibitory effect mediated by ETH was shown by the ability of ETH treatment to significantly decrease the expression of multiple ARE-dependent genes, including HO1 (FIG. 4A), GCLM (FIG. 4B), and sulfiredoxin (SRX, FIG. 4C) in THP-1 cells under basal conditions. Likewise, confirmation of the ability of an Nrf2 activator to modulate the Nrf2-ARE-inhibitory activity of a drug having Nrf2-ARE-inhibitory activity (e.g., ETH) was shown by an increased expression of multiple ARE-dependent genes, including HO1 (FIG. 4A), GCLM (FIG. 4B), and sulfiredoxin 1 (SRX, FIG. 4C) in THP-1 cells treated with both the Nrf2 activator and the drug having Nrf2-ARE-inhibitory activity, as compared to expression of the ARE-dependent genes in cells treated with the drug having Nrf2-ARE-inhibitor activity alone (e.g., under basal conditions). Thus, it is a surprising discovery that known antitubercular drugs isoniazid and ethionamide demonstrate Nrf2-ARE inhibitory activity.

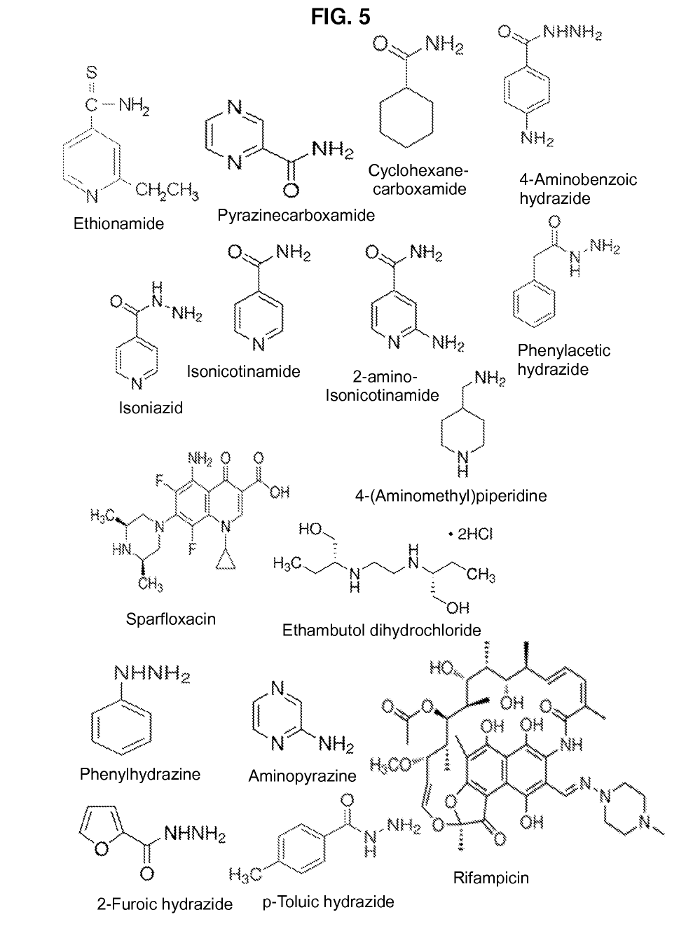

By using the same methods, it was demonstrated that (a) known antitubercular agents other than isoniazid and ethionamide, including ethambutol dihydrochloride, rifampicin, and sparfloxacin, surprisingly demonstrate Nrf2-ARE inhibitory activity as demonstrated by the concentration-dependent inhibitory effect on ARE-luciferase activity under basal conditions (Table 1). In addition, a number of other compounds represented by either Formula I or Formula II, particularly heterocyclic compounds having a hydrazide side chain or carboxamide side chain, including 4-aminobenzoic hydrazide, aminopyrazine, 2-furoic hydrazide, cyclohexane-carboxamide, phenylhydrazine, phenylacetic hydrazide, pyrazinecarboxamide, and p-toluic hydrazide, were discovered inhibit Nrf2 activity as demonstrated by the concentration-dependent inhibitory effect on ARE-luciferase activity under basal conditions and iAs³⁺-treated or tBHQ-treated conditions (Table 1). As apparent from FIG. 5 showing the chemical structure of these newly discovered Nrf2 inhibitors, many of these compounds are represented by Formula I or Formula II, particularly heterocyclic compounds having a hydrazide side chain or carboxamide side chain, suggesting a structure-function relationship between such chemical representation and the ability to inhibit Nrf2-ARE activity. Based on this discovery, and structure-function relationship, additional compounds represented by Formula I or Formula II may be screened for activity as Nrf2 inhibitors. Illustrative examples of such compounds may include pyrrole-2 carboxamide, and pyrrole-2 hydrazide, or other compounds consisting of five- or six-membered rings that comprise a hydrazide side chain or carboxamide side chain.

TABLE 1

| | | Concentration | | Concentration | |
|---|---|---|---|---|---|
| | | tested & showing | | tested & showing | |
| | | inhibition on | | inhibition on | |
| Compound | CAS number | basal ARE activity | | inducible ARE activity | |
| 4-Aminobenzoic hydrazide | 5351-17-7 | 1 | mM | 0.1-1 | mM |
| Aminopyrazine | 5049-61-6 | 1.0-10 | mM | 1.0-10 | mM |
| Cyclohexanecarboxamide | 1122-56-1 | 10 | mM | 0.1-10 | mM |
| Ethambutol dihydrochloride | 1070-11-7 | 10 | mM | 0.1-10 | mM |
| Ethionamide | 536-33-4 | 0.1-2 | mM | 0.1-2 | mM |
| 2-Furoic hydrazide | 3326-71-4 | 10 | mM | 0.1-10 | mM |
| Phenylhydrazine | 100-63-0 | 1-10 | mM | 0.1-10 | mM |
| Isoniazid | 54-85-3 | 1-50 | mM | 1-50 | mM |
| Isonicotinamide | 1453-82-3 | 1-50 | mM | 1-50 | mM |
| 2-Amino-isonicotinamide | 13538-42-6 | 1-50 | mM | 1-50 | mM |
| Phenylacetic hydrazide | 937-39-3 | 10 | mM | 1-10 | mM |
| Pyrazinecarboxamide | 98-96-4 | 1-10 | mM | 1-10 | mM |
| (including pyrazinamide) | | | | | |
| Rifampicin | 13292-46-1 | 0.5 | mM | 0.1-0.5 | mM |
| Sparfloxacin | 110871-86-8 | 0.1-1 | mM | 0.1-1 | mM |
| p-Toluic hydrazide | 3619-22-5 | 10 | mM | 0.1-1 | mM |

| | | | | |
|---|---|---|---|---|
| 4-(Aminomethyl)piperidine | 7144-05-0 | — | 0.1-1 | mM |

### Example 2

In this Example, provided is an illustration of combination therapy comprising use of an Nrf2 inhibitor (including a salt thereof) with at least one known antitubercular drug; and more particularly providing an Nrf2 inhibitor in a medically effective amount, and a known antitubercular drug in a medically effective amount to treat Mycobacterium tuberculosis infection or tuberculosis in an individual in need. The inventors of the present application surprisingly discovered that antitubercular drugs having a primary mechanism of action other than Nrf2-ARE-inhibitory activity, also were discovered to have Nrf2-ARE-inhibitory activity (e.g., rifampicin, isoniazid, pyrazinamide, sparfloxacin, ethambutol dihydrochloride, ethionamide, or salt thereof). Also, the inventors of the present application surprisingly discovered chemical signatures for Nrf2-ARE-inhibitory activity. Thus, provided are compounds represented by Formula I or Formula II as Nrf2 inhibitors, and the new use of one more of such compounds comprising an Nrf2 inhibitor to treat Mycobacterium infection such as M. tuberculosis, M. bovis, or M. africanum, or a Mycobacterium species that is environmental or opportunistic and that causes opportunistic infections such as lung infections in immune compromised hosts (e.g., patients with AIDS), e.g., BCG, M. avium, M. intracellulare, M. celatum, M. genavense, M. haemophilum, M. kansasii, M. simiae, M. vaccae, M. fortuitum, and M. scrofulaceum.

Such a compound may be used in combination with a known antitubercular drug to treat an individual infected with Mycobacterium. Thus, provided is a method of administering an Nrf2 inhibitor in a medically effective amount for treating a Mycobacterium infection in an individual infected with Mycobacterium. In one aspect of this method, the Mycobacterium infection comprises M. tuberculosis infection. The method may comprise selecting an individual having such disease to be treated or to whom the composition is to be administered (e.g., to an individual in need thereof), followed by administration of the compound or composition of the invention. The compound is administered to an individual (a mammal, such as a human) selected for treatment because the individual has one or more of mycobacterial infection or tuberculosis caused by mycobacterial infection (as an individual in need thereof). The compound may be administered in the form of a pharmaceutical composition. The pharmaceutical composition may further comprise a pharmaceutically acceptable carrier, or a pharmaceutically acceptable salt of the Nrf2 inhibitor. Also provided is an Nrf2 inhibitor of the invention, used in the manufacture of a medicament for treating one or more of mycobacterial infection or tuberculosis caused by mycobacterial infection.

If the medicament or pharmaceutical composition comprises, as the sole therapeutically active agent or pharmaceutically active agent, an Nrf2 inhibitor, then in one aspect of the invention the Nrf2 inhibitor may comprise a non-naturally occurring compound. In this example, an Nrf2 inhibitor is administered by itself (e.g., is the sole therapeutic agent or pharmaceutically active agent in a pharmaceutical composition, which may further comprise a pharmaceutically acceptable carrier), and is not combined with at least one known antitubercular drug or second Nrf2 inhibitor to produce such pharmaceutical composition or medicament. In another example, in one aspect, when an Nrf2 inhibitor is combined together with one or more of a second Nrf2 inhibitor or known antitubercular drug in producing a pharmaceutical composition or medicament (comprising a combination of the Nrf2 inhibitor and one or more of a second Nrf2 inhibitor or known antitubercular drug), such Nrf2 inhibitor may be selected from a non-naturally occurring compound or a naturally occurring compound.

The mode of administration of an compound or composition comprising an Nrf2 inhibitor according to the invention to an individual (such as a human) in need of such composition or compound may be any mode known in the art to be suitable for delivering a pharmaceutical composition, and particularly suitable for inhibiting Nrf2-ARE activity in an M. tuberculosis infection or in tuberculosis caused by mycobacterial infection, and may include but is not limited to, intravenously, intraperitoneally, orally, subcutaneously, intramuscularly, intranasally or other mode of inhaled therapy, transdermally, by perfusion, and by peristaltic techniques. Provided herein is a method for treating M. tuberculosis infection comprising administering to an individual in need thereof one or more Nrf2 inhibitors in a medically effective amount by itself, or in combination with a known antitubercular drug, to inhibit Nrf2-ARE activity in cells of the treated individual. In a method of combination therapy, one or more Nrf2 inhibitors and a known antitubercular drug may be administered concurrently, sequentially, or in regimen alternating between an Nrf2 inhibitor and one or more known antitubercular drugs. Such combination therapy may optionally include one or more additional therapeutic agents for treating the disease caused by Mycobacterial infection. The structure of such additional therapeutic agents, Nrf2 inhibitor (as described herein), known antitubercular drug, and their generic or trademark names, are readily available to those skilled in the art, such as from the standard compendium of drugs (e.g., The Merck Index) or from the applicable pharmaceutical company's web site, as well as dosages applicable for treatment (see also The Physician's Desk Reference). Alternatively, the doses and dosage regimen of an additional therapeutic agent, known antitubercular drug, and an Nrf2 inhibitor used in accordance with the invention in combination therapy, can be determined by a physician, taking into account the medical literature, the health, age and sex of the patient, the disease or condition or disorder to be treated, the mode of administration and dosing schedule, and other relevant considerations. Generally, dosages of such compounds can range from about 0.1 mg to 1000 mg per day, with more specific dosages dependent on the aforementioned factors.

Accordingly, also provided herein is a pharmaceutical composition or medicament comprising a medically effective amount of an Nrf2 inhibitor, in combination with a medically effective amount of one or more known antitubercular drugs; and optionally further comprising a pharmaceutically acceptable carrier. In accordance with this invention, a medically effective amount of one or more Nrf2-ARE-inhibitors and a medically effective amount of one or more known antitubercular drugs can be used for the preparation of a medicament or pharmaceutical composition useful for treating mycobacterial infection in individuals, particularly in humans. With respect to use with known antitubercular drugs, an Nrf2 inhibitor may be used in combination with more than one known antitubercular drugs in the standard “short” course treatment for tuberculosis comprising isoniazid, rifampicin (also known as rifampin), pyrazinamide, and optionally ethambutol, for two months, then with isoniazid and rifampicin alone for an additional four months. If the tuberculosis is latent, an Nrf2 inhibitor may be used with one known antitubercular drug; for example, the current standard for such treatment is six to nine months of isoniazid alone. Thus, an Nrf2 inhibitor may be used with isoniazid in a treatment regimen lasting for six to nine months or in a duration that can be determined by a physician, taking into account one or more factors such as the health and age of the individual being treated, the severity of the disease to be treated, measurable responses to treatment, and other relevant considerations.

In combination therapy, an Nrf2 inhibitor and a known antitubercular drug may be administered concurrently, sequentially, or in regimen alternating between one or more Nrf2 inhibitors and one or more known antitubercular drugs. The combination therapy according to the invention includes, but is not limited to, administration of a single pharmaceutical composition or dosage formulation which comprises both the Nrf2 inhibitor and a known antitubercular drug; as well as administration of a first pharmaceutical formulation or composition comprising an Nrf2 inhibitor, and a second pharmaceutical formulation or composition comprising a known antitubercular drug.

Where the combination therapy comprises administration of a single pharmaceutical composition or dosage formulation comprising both an Nrf2 inhibitor and at least one known antitubercular drug, and where the single pharmaceutical dosage formulation is administered orally, the single pharmaceutical dosage formulation can be administered to an individual in one oral composition, such as a tablet or capsule; or in one inhaled composition (e.g., propellant-based inhalation, and nasal aerosols). For example, an Nrf2 inhibitor in a medically effective amount, and a known antitubercular drug in a medically effective amount, may be formulated together in one oral formulation such as a single tablet or capsule. As an illustration, isoniazid is available in a medically effective amount such as 100 mg and 300 mg tablets for oral administration. Thus in this illustration an oral composition, as a single pharmaceutical dosage formulation may comprise a medically effective amount of isoniazid (e.g., 100 mg or 300 mg) and a medically effective amount of an Nrf2 inhibitor in a single tablet or capsule. Tablets or capsules with a lesser dosage amount of each may be produced but would require multiple tablets or capsules in a single oral administration. Optionally, the oral composition may further comprise an additional known antitubercular drug. For example, when used in combination with an Nrf2 inhibitor and isoniazid for treatment of tuberculosis, the oral composition may further comprise one or more of 300 to 600 mg rifampin, or 500 mg to 2 g of pyrazinamide, or 100 mg to 400 mg of bedaquiline. Alternatively, a medically effective amount of an Nrf2 inhibitor may be formulated in a single tablet or capsule with a medically effective amount of one or more known antitubercular drugs other than isoniazid. Yet another alternative is to formulate the single pharmaceutical dosage formulation for inhaled administration (e.g., comprising an aerosol, particulate inhalation medicament, or with an inhalant propellant known in the art).

As previously described herein, the tablet or capsule may be formulated with inactive ingredients including, but not limited to, colloidal silicon dioxide, lactose monohydrate, pregelatinized starch, stearic acid, sodium, microcrystalline cellulose, silicified microcrystalline cellulose, croscarmellose, talc, silica colloidal silicon dioxide, magnesium stearate, triethyl citrate, methacrylic acid copolymer-Type A, methacrylic acid copolymer dispersion, simethicone emulsion, sodium lauryl sulphate, polysorbate 80, and combinations thereof. Also provided herein is a kit for combination therapy for treating Mycobacterial infection, the kit comprising an Nrf2 inhibitor and one or more known antitubercular drugs, together with packaging for same. The kit can include one or more separate containers, dividers or compartments and, optionally, informational material such as instructions for administration. For example, each of an Nrf2 inhibitor and one or more known antitubercular drugs (or various combinations thereof) can be contained in a bottle, vial, or syringe, and the informational material can be contained in a plastic sleeve or packet or provided in a label. In some embodiments, the kit may comprise a plurality (e.g., a pack) of individual containers, each containing one or more unit dosage forms of a compound comprising an Nrf2 inhibitor, one or more known antitubercular drugs, or a combination thereof. For example, the kit can include a plurality of foil packets, or blister packs, each containing a single unit dose of a compound described herein or any of the various combinations thereof.

What is claimed is:

1. A pharmaceutical composition comprising an Nrf2 inhibitor comprising a compound represented by Formula I or Formula II; wherein the Nrf2 inhibitor is in a medically effective amount in the pharmaceutical composition to inhibit Nrf2-ARE activity when administered to an individual in need thereof; wherein Nrf2 is comprised of an amino acid sequence comprising SEQ ID NO:1 including isoforms thereof; wherein the Nrf2 inhibitor is a non-naturally occurring compound when the pharmaceutical composition consists of the Nrf2 inhibitor as a sole therapeutic agent, and is either a naturally occurring compound or a non-naturally occurring compound when the pharmaceutical composition comprises the Nrf2 inhibitor in combination with one or more of (a) a known antitubercular drug or (b) a second Nrf2 inhibitor; wherein Formula I is 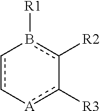
wherein A is N or C; B is N or C; R1 or R2 or R3 are each independently selected from H, (C₁-C₆)alkyl, CONH₂, CONHNH₂, CSNH₂, SO₂NH₂, NH₂, NHNH₂, CHCHCONH₂, CHCHCONHNH₂, or COCH₃; wherein at least one of R1, R2, and R3 is selected from CONH₂, CONHNH₂, CSNH₂, SO₂NH₂, NH₂, NHNH₂, CHCHCONH₂, or CHCHCONHNH₂; with dashed lines representing optional double bonds; and a pharmaceutically acceptable salt thereof; with the proviso that the compound of Formula I is not a known antitubercular drug;
and wherein Formula II is 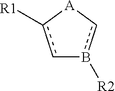
wherein A is O or N; B is N or C; R1 is selected from CONH₂, CONHNH₂, CSNH₂, SO₂NH₂, NH₂, NHNH₂, CHCHCONH₂, or CHCHCONHNH₂; R2 is absent if B is NH; if B is C, R2 is absent or selected from CH₃, CH₂CH₃, NH₂, or NHNH₂; with dashed lines representing optional double bonds; and a pharmaceutically acceptable salt thereof; with the proviso that the compound of Formula II is not a known antitubercular drug.

2. The pharmaceutical composition of claim 1, which contains a known antitubercular drug in a medically effective amount in combination with one or more Nrf2 inhibitors.

3. The pharmaceutical composition according to claim 1, which is administered concurrently, sequentially, or in a regimen of alternating dose, in treatment of one or more of mycobacterial infection or tuberculosis caused by mycobacterial infection.

4. The pharmaceutical composition of claim 1, further comprising a pharmaceutically acceptable carrier

5. Use of an Nrf2 inhibitor in a medically effective amount to inhibit Nrf2-ARE activity, in combination with at least one known antitubercular drug, in treatment of one or more of mycobacterial infection or tuberculosis caused by mycobacterial infection; wherein Nrf2 is comprised of an amino acid sequence comprising SEQ ID NO:1 including isoforms thereof; wherein the Nrf2 inhibitor is (i) selected from the group consisting of compounds represented by Formula I or Formula II, (ii) a non-naturally occurring compound for administration by itself, and (iii) either a naturally occurring compound or a non-naturally occurring compound when combined together, to form a pharmaceutical composition, with one or more of (a) a known antitubercular drug, or (b) a second Nrf2 inhibitor; with Formula I as 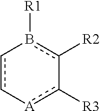
wherein A is N or C; B is N or C; R1 or R2 or R3 are each independently selected from H, (C₁-C₆)alkyl, CONH₂, CONHNH₂, CSNH₂, SO₂NH₂, NH₂, NHNH₂, CHCHCONH₂, CHCHCONHNH₂, or COCH₃; wherein at least one of R1, R2, and R3 is selected from CONH₂, CONHNH₂, CSNH₂, SO₂NH₂, NH₂, NHNH₂, CHCHCONH₂, or CHCHCONHNH₂; with dashed lines representing optional double bonds; and a pharmaceutically acceptable salt thereof; with the proviso that the compound of Formula I is not a known antitubercular drug;
and with Formula II as 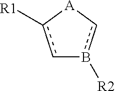
wherein A is O or N; B is N or C; R1 is selected from CONH₂, CONHNH₂, CSNH₂, SO₂NH₂, NH₂, NHNH₂, CHCHCONH₂, or CHCHCONHNH₂; R2 is absent if B is NH; if B is C, R2 is absent or selected from CH₃, CH₂CH₃, NH₂, or NHNH₂; with dashed lines representing optional double bonds; and a pharmaceutically acceptable salt thereof; with the proviso that the compound of Formula II is not a known antitubercular drug.

6. A method for treatment of a disease comprising one or more of a mycobacterial infection, or tuberculosis caused by mycobacterial infection, the method comprising administering to an individual in need thereof a medically effective amount of a pharmaceutical composition according to claim 1.

7. The method of claim 6, wherein the pharmaceutical composition further comprises a pharmaceutically acceptable carrier.

8. The method of claim 6, wherein the pharmaceutical composition further comprises at least one antitubercular drug in a medically effective amount.

9. The method of claim 6, wherein the pharmaceutical composition is administered concurrently, sequentially, or in a regimen of alternating dose, in a treatment further comprising administration of at least one known antitubercular drug.

10. The method of claim 6, wherein the mycobacterial infection comprises an infection comprising M. tuberculosis.

11. A method for treating a mycobacterial infection comprising administering to an individual in need thereof an Nrf2 inhibitor in a medically effective amount, and one or more known antitubercular drugs in a medically effective amount; wherein Nrf2 is comprised of an amino acid sequence comprising SEQ ID NO:1 including isoforms thereof; wherein the Nrf2 inhibitor is (i) selected from the group consisting of compounds represented by Formula I or Formula II, (ii) a non-naturally occurring compound for administration by itself, and (iii) either a naturally occurring compound or a non-naturally occurring compound when combined together, to form a pharmaceutical composition, with one or more of (a) a known antitubercular drug, or (b) a second Nrf2 inhibitor; with Formula I as 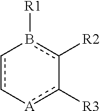
wherein A is N or C; B is N or C; R1 or R2 or R3 are each independently selected from H, (C₁-C₆)alkyl, CONH₂, CONHNH₂, CSNH₂, SO₂NH₂, NH₂, NHNH₂, CHCHCONH₂, CHCHCONHNH₂, or COCH₃; wherein at least one of R1, R2, and R3 is selected from CONH₂, CONHNH₂, CSNH₂, SO₂NH₂, NH₂, NHNH₂, CHCHCONH₂, or CHCHCONHNH₂; with dashed lines representing optional double bonds; and a pharmaceutically acceptable salt thereof; with the proviso that the compound of Formula I is not a known antitubercular drug;
and with Formula II as 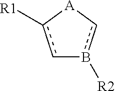
wherein A is O or N; B is N or C; R1 is selected from CONH₂, CONHNH₂, CSNH₂, SO₂NH₂, NH₂, NHNH₂, CHCHCONH₂, or CHCHCONHNH₂; R2 is absent if B is NH; if B is C, R2 is absent or selected from CH₃, CH₂CH₃, NH₂, or NHNH₂; with dashed lines representing optional double bonds; and a pharmaceutically acceptable salt thereof; with the proviso that the compound of Formula II is not a known antitubercular drug.

12. The method according to claim 11, wherein an Nrf2-ARE-inhibitor and one or more known antitubercular drugs are administered together in a single pharmaceutical composition.

13. The method of claim 11, wherein the pharmaceutical composition further comprises a pharmaceutically acceptable carrier.

14. A kit for combination therapy comprising an Nrf2 inhibitor and one or more known antitubercular drugs, together with packaging and one or more separate containers; wherein Nrf2 is comprised of an amino acid sequence comprising SEQ ID NO:1 including isoforms thereof; wherein the Nrf2 inhibitor is (i) selected from the group consisting of compounds represented by Formula I or Formula II, (ii) a non-naturally occurring compound for administration by itself, and (iii) either a naturally occurring compound or a non-naturally occurring compound when combined together, to form a pharmaceutical composition, with one or more of (a) a known antitubercular drug, or (b) a second Nrf2 inhibitor; with Formula I as 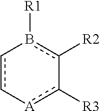
wherein A is N or C; B is N or C; R1 or R2 or R3 are each independently selected from H, (C₁-C₆)alkyl, CONH₂, CONHNH₂, CSNH₂, SO₂NH₂, NH₂, NHNH₂, CHCHCONH₂, CHCHCONHNH₂, or COCH₃; wherein at least one of R1, R2, and R3 is selected from CONH₂, CONHNH₂, CSNH₂, SO₂NH₂, NH₂, NHNH₂, CHCHCONH₂, or CHCHCONHNH₂; with dashed lines representing optional double bonds; and a pharmaceutically acceptable salt thereof; with the proviso that the compound of Formula I is not a known antitubercular drug;
and with Formula II as 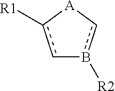
wherein A is O or N; B is N or C; R1 is selected from CONH₂, CONHNH₂, CSNH₂, SO₂NH₂, NH₂, NHNH₂, CHCHCONH₂, or CHCHCONHNH₂; R2 is absent if B is NH; if B is C, R2 is absent or selected from CH₃, CH₂CH₃, NH₂, or NHNH₂; with dashed lines representing optional double bonds; and a pharmaceutically acceptable salt thereof; with the proviso that the compound of Formula II is not a known antitubercular drug.
